(19) United States
(12) Patent Application Publication
(10) Pub. No.: US 2022/0323715 A1
(43) Pub. Date: Oct. 13, 2022

(54) OPTIMIZED CATHETER USAGE

(71) Applicant: Hill-Rom Services, Inc., Batesville, IN (US)

(72) Inventors: Susan Kayser, Batesville, IN (US); Mary L. Pfeffer, Batesville, IN (US); Jennifer Marie Rizzo, Batesville, IN (US)

(21) Appl. No.: 17/657,663

(22) Filed: Apr. 1, 2022

Related U.S. Application Data
(60) Provisional application No. 63/173,671, filed on Apr. 12, 2021.

(51) Int. Cl. A61M 25/00; A61B 5/00; G16H 40/63; A61B 5/20

(52) U.S. Cl. CPC A61M 25/0017; A61B 5/6852; G16H 40/63; A61B 5/207; A61M 2205/18; A61B 5/746

(57) ABSTRACT

An example system for assessing catheterization risk can include: a processor; and memory with instructions which, when executed by the processor, cause the processor to: receive information relating to a current state of a patient; perform an assessment of risk associated with catheterization of the patient; and provide a recommendation for the catheterization based upon the assessment of risk

### BACKGROUND

Hospitals are increasingly expected to reduce the number of Catheter Associated Urinary Tract Infections (CAUTIs) caused by catheter use. There are a few ways to decrease the rate of CAUTIs, including using indwelling (aka Foley) catheters less frequently, removing them sooner after surgeries, and more frequent perineal cleaning (aka peri care).

### SUMMARY

In general terms, the present disclosure relates to the optimization of catheter usage. Tracking and notification of that usage can also be provided.

One aspect relates to a system for assessing catheterization risk, which can include: a processor; and memory encoding instructions which, when executed by the processor, cause the processor to: receive information relating to a current state of a patient; perform an assessment of risk associated with catheterization of the patient; and provide a recommendation for the catheterization based upon the assessment of risk.

Another aspect relates to a method for assessing risk associated with catheterization, which can include: receiving information relating to a current state of a patient; performing an assessment of risk associated with catheterization of the patient; and providing a recommendation for the catheterization based upon the assessment of risk.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates a patient care system.

FIG. 2 illustrates an example server of the patient care system of FIG. 1.

FIG. 3 illustrates an example method of optimizing the usage of catheters by the server of FIG. 2.

FIG. 4 illustrates an example user interface generated on a mobile device of the patient care system of FIG. 1.

FIG. 5 illustrates example components of the server of FIG. 2.

### DETAILED DESCRIPTION

In general terms, the present disclosure relates to the optimization of catheter usage. Tracking and notification of that usage can also be provided.

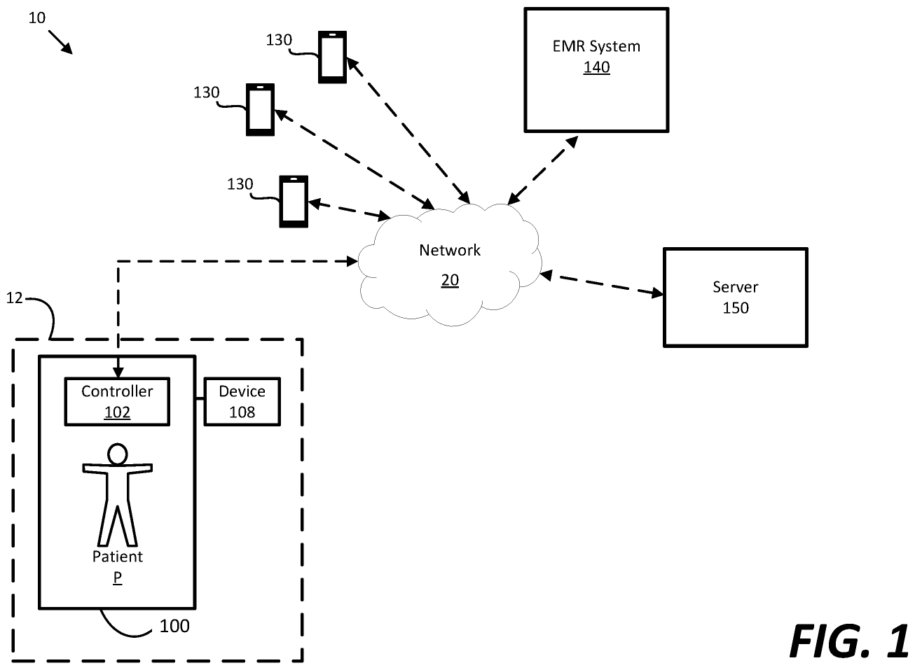

FIG. 1 schematically illustrates a patient care system 10 that optimizes the use of catheterization for a patient P within a patient environment 12. Generally, the patient care system 10 includes a network 20, mobile devices 130, an electronic medical record (EMR) system 140, and a server 150.

In some illustrative examples, the patient environment 12 is a patient room within a healthcare facility such as a hospital, a surgical center, a nursing home, a long term care facility, and the like. In other examples, the patient environment 12 is the patient P's home.

The patient care system 10 includes the network 20 that receives information associated with catheterization usage within the patient environment 12. This information can be acquired from and/or stored with an electronic medical record (EMR) system 140 (alternatively termed electronic health record (EHR)) of the patient P. The network 20 communicates information for the patient P between a controller 102, a device 108, the mobile devices 130, the EMR system 140, and the server 150.

One or more wireless communications protocols including Wi-Fi, Bluetooth, Z-Wave, Zigbee, and the like, and/or one or more wired communications protocols including Ethernet, USB, and like can be used to connect the controller 102 and the patient support apparatus 100. These communications protocols provide an Internet of Things (TOT) network within the patient environment 12 that is private and secure. This can improve communication among the devices located within the patient environment 12, to authenticate, authorize, and associate the devices with each other, and perform coordinated operations. These functionalities, including authentication and authorization, can be distributed and performed in the IOT network with or without using a dedicated edge-computational resources of the healthcare facility.

The network 20 can communicate changes in the patient P's status directly to a plurality of mobile devices 130. Each mobile device 130 can be carried by a caregiver in the healthcare facility. An application installed on each mobile device 130 allows each caregiver to use the mobile device 130 to monitor notifications regarding the status of the patient P.

The network 20 can also communicate changes in the patient P's status directly to the server 150 which is an area in a healthcare facility, such as a hospital or nursing home, where caregivers such as nurses' and other staff work when not working directly with the patient P such as where they can perform administrative tasks. The server 150 includes one or more computing devices connected to the network 20.

As shown in FIG. 1, the patient environment 12 includes the patient support apparatus 100 on which the patient P can rest. In certain examples, the patient support apparatus 100 is a bed. Alternatively, the patient support apparatus 100 can be a chair, a recliner, a stretcher, surgical table, or any other apparatus on which a patient can rest.

The patient support apparatus 100 includes devices that detect various aspects associated with catheterization of the patient P while the patient P rests on the patient support apparatus 100. For example, the patient support apparatus 100 can be configured to capture information associated with the use of a catheter for the patient P.

The controller 102 is associated with the patient support apparatus 100. The controller 102 is programmed with instructions stored on a memory of the controller 102. When executed by a processor of the controller 102, the instructions cause the controller 102 to provide various information and aspects associated with the catheterization of the patient P, as described further below.

In some examples, the controller 102 of the patient support apparatus 100 transmits bed data, vital signs data, and catheter monitor data. For instance, as described in U.S. Pat. No. 10,363,183 to Ribble, which is hereby incorporated by reference, when catheter tubing is connected to a controller 102 integrated into the patient support apparatus 100, the controller initiates a countdown timer pertaining to removal of catheter tubing for the patient P is displayed on the mobile device 130.

For instance, the controller 102 is operable to implement a countdown timer to indicate an amount of time before catheter tubing should be removed from the patient (referred to herein as “countdown to removal”). In some embodiments, the countdown timer is activated in response to a signal to the controller 102 indicating at least one of the following: the catheter monitor 60 being turned on, a catheter tube 62 or 66 being attached to the catheter monitor, and urine flowing through the catheter monitor 60.

In some embodiments, an initial period of time at which the countdown timer is set may be no longer than 24 hours. It is contemplated by this disclosure that a notification may be initiated by controller 102 and/or the server 150 in response to expiration of the countdown timer. Alternatively or additionally, a notification may be initiated by controller 102 a preset amount of time prior to the expiration of the countdown timer. It is contemplated by this disclosure that the countdown timer may be displayed on various user interfaces, such as the mobile device 130.

In the illustrative example, the controller 102 or server 150 sends a notification to a caregiver in response to expiration of the countdown timer or upon a preset amount of time prior to the expiration of the countdown timer being reached.

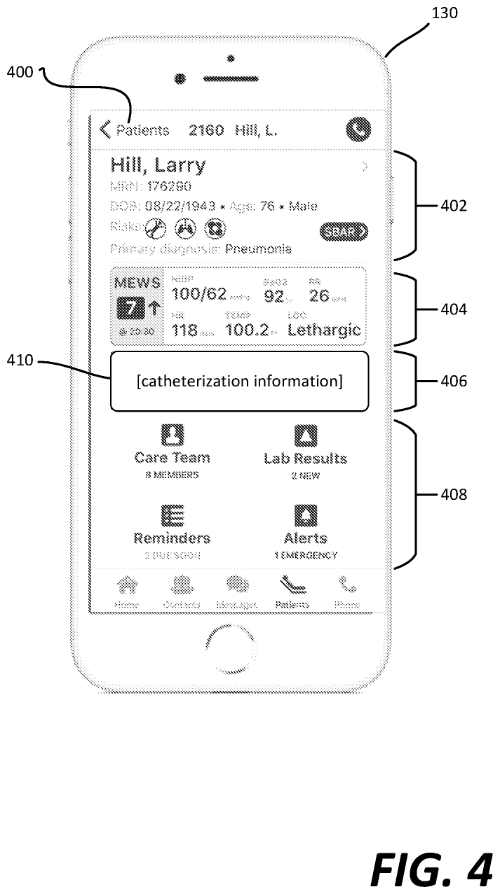

In some embodiments, a caregiver enters (e.g., on the user interface described in reference to FIG. 4 below) a countdown time of their choosing which may be greater than or less than the 24 hour default countdown time. Alternatively or additionally, the caregiver is able to use one or more of the mobile devices 130 to enter other times at which the catheter is to be checked prior to countdown-to-removal time expiration (e.g., checking every 6 hours or every 8 hours, just to name a couple possible examples). Thus, the caregiver is able to enter pre-determined time limits and to be notified as to when the catheter should be checked, changed or removed.

The times entered by the caregiver for catheter removal, or automatically determined by the controller 102 or the server 150, is based on the type of catheter being used, the type of patient with which the catheter is being used, or both. For example, indwelling catheters may need to be changed more frequently than external (e.g., condom) catheters or less frequently than short term or intermittent catheters. Catheters may need to be changed more frequently for children than for adults or more frequently when the patient has undergone bladder surgery rather than knee surgery, just to name a couple of examples.

The type of catheter is able to be determined based on the catheter ID read by the controller 102 and the type of patient is able to be determined based on a patient ID or otherwise stored in RTLS server and/or the server 150 and/or some other server of the associated system.

In addition to notifications about removal, the controller 102 and the server 150 can be programmed to provide other notifications. For instance, the controller 102 can be programmed to monitor for caregiver handwashing compliance in connection with the catheterization.

For example, the controller 102 can be programmed to determine whether the caregiver attaching the catheter tubing has used a hand hygiene system dispenser within a threshold amount of time prior to connecting the catheter tubing. In some embodiments, the threshold amount of time for handwashing may be selectable. A notification is generated by the controller 102 if the catheter tubing is connected and the caregiver did not use one of the hand hygiene system dispensers within the threshold amount of time prior to connecting the catheter tubing. The notification may include a message displayed at one or more of the mobile devices 130 and the server 150.

The device 108 can be programmed to provide vital signs information associated with the patient P, as described herein. This vital signs information can be stored in the EMR system 140 and/or sent to the controller 102.

In addition to monitoring aspects associated with the introduction, dwell time, and removal of the catheter for the patient P, the patient care system 10 is also configured to manage other risks associated with the catheterization. These risks can generally account for other factors that impact a catheterization, such as one or more of: (i) the specific patient demographics and medical history for the patient; (ii) current medical state of the patient; (iii) type of procedure(s) performed on the patient; and (iv) medications provided to the patient.

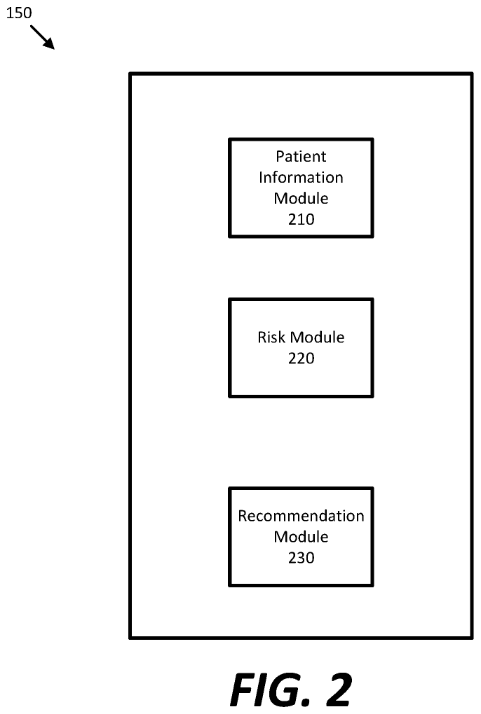

For example, referring now to FIG. 2, additional details about the server 150 are shown. The server 150 is programmed to manage the risks associated with the catheterization of the patient P. In this example, the server 150 includes a patient information module 210, a risk module 220, and a recommendation module 230.

The patient information module 210 is programmed to access information associated with the patient P from the device 108 and the EMR system 140. The device 108 can monitor and report vital signs associated with the patient P, such as pulse oximetry, non-invasive blood pressure (NIBP), temperature, end-tidal CO2 (EtCo2), respiration and more. A change in vital signs for the patient P can indicate a likely infection and therefore a possible infection. One example of a vital signs monitor is the Connex® Vital Signs Monitor from Welch Allyn Inc.

Other devices can also be used to obtain data about the patient P. For example, the device 108 can also include the WatchCare™ Incontinence Management System from Hillrom. For example, a smart incontinence pad can be used to determine if the patient P is incontinent. The smart pad can therefore indicate that the catheter may be leaking or if there is a fecal incontinence event, which can increase the risk of infection. In another example, the PureWick™ urine collection system from Liberator Medical Supply, Inc.

This information from the EMR system 140 can include demographic information about the patient P. Such demographic information includes age, sex, race, ethnicity, etc. This information can also include the medical history for the patient P. For instance, medical history information can include prior diseases, illnesses, surgeries, allergies, weight, etc. Some disease states, such as diabetes, can increase the likelihood for urinary tract infections associated with catheterization. Further, prior medical history, such as previous urinary retention, can be an indicator for future problems associated with catheterization.

The information can also include the current medical state for the patient P. For example, the current medical state can include vital signs data from the vital signs monitor 108 and/or information from the EMR system 140, such as a present disease, trauma, chronic illness, or other reason prompting the hospitalization of the patient P. The current medical state can also dictate the catheterization of the patient P. For instance, the current medical state can include a blockage or injury to the urethra, an enlarged prostate in males, kidney or bladder stones, etc.

Further, the information can include the type of procedure(s) performed on the patient P. For example, having surgery on any part of the urinary tract, including the kidneys, ureters, bladder, and urethra are also a risk factor for a urinary tract infection. Additionally, being immobilized after surgery, which is common after trauma and orthopedic procedures including some joint surgeries, will increase the probability of a problem.

For example, surgery to repair certain conditions, like a broken hip, can require catheterization due to the difficulty for the patient P to use normal processes to urinate. Further, the conditions needed for surgery, such as anesthesia, may require catheterization.

Further, some procedures require certain processes or aftercare that can increase risk. For instance, some procedures required surgical briefs to be applied to the patient P after the procedures. These briefs can increase the risk of infection, as the briefs can hold feces close to the urinary track. Other configurations are possible.

In addition, the information from the EMR system 140 can include any medications that are provided to the patient P. For instance, some medications can cause various complications, such as diarrhea. Such a condition can impact the likelihood of a urinary tract infection due to the bacteria associated with the diarrhea. Other configurations are possible.

While the example provides the patient information module 210 that is programmed to obtain the noted information, some or all of the information can be obtained from other sources. For instance, the current medical state information for the patient P can be obtained from other systems associated with the patient care system 10, such as a vital signs monitor connected to the patient P. Further, some of this information can be provided manually, such as through input by the caregiver. For instance, the caregiver can provide status information for the patient P to the server 150.

The example risk module 220 is programmed to conduct a risk assessment associated with the catheterization of the patient P based upon the information from the patient information module 210. In some examples, the risk module 220 executes one or more algorithms used to assess the risk of catheterization of the patient P.

For example, the risk module 220 can employ an algorithm to assess the risk associated with catheterization. One example pseudocode for such an algorithm follows as Equation 1.

Risk score=[demographics]+[medical state]+[procedure]+[medications] (1)

In Equation 1, a risk score is quantified as a combination of the various information obtained by the patient information module 210.

For example, the patient P can be an 80 year old female with incontinence that has a history of urinary tract infections. The age (older), gender (female) and history of infections will cause the risk score to increase.

In some examples, artificial intelligence is used by the risk module 220 to quantify the risk score. For instance, a machine learning algorithm can be trained using data from a large number of patients. The machine learning can then be leveraged to determine the risk score for the patient P based upon the noted information.

In some examples, the risk module 220 is programmed to automatically conduct an assessment for the patient P after a triggering event. For instance, the risk module 220 can be programmed to automatically assess risk after the patient P undergoes a procedure that typically requires catheterization, such as surgery on any part of the urinary tract.

In another example, the risk module 220 is automatically triggered when an order is placed to catheterize the patient P. In yet other examples, the risk module 220 can be manually initiated. For instance, the caregiver can manually initiate (from the mobile device 130) the risk assessment by the risk module 220 when contemplating catheterization of the patient P.

Once the assessment of risk is complete, the risk module 220 communicates the assessment to the recommendation module 230. The recommendation module 230 is programmed to provide one or more recommendations to the caregiver based upon the assessment by the risk module 220.

For example, the recommendation module 230 can be programmed to assess the risk score from the risk module 220 by applying the risk score to a threshold. If the risk score exceeds the threshold, the recommendation module 230 is programmed to indicate the risk associated with catheterization.

The indication can take multiple forms. In one example, the recommendation module 230 simply provides a notification to the caregiver that catheterization of the patient P presents a heightened risk of urinary tract infection. This notification can be delivered to the mobile device 130 (see FIG. 4).

In another example, the recommendation module 230 can also provide a recommendation for a different catheterization option or process. For instance, assuming that a standard indwelling catheter (e.g., Foley catheter) is ordered for the patient P. The recommendation module 230 uses the risk score to determine if the patient P is at a higher risk for a urinary tract infection. If so, the recommendation module 230 notifies the caregiver and provides a recommendation for a different catheterization option, such as an external catheter (e.g., condom catheter) or short-term catheter (e.g., intermittent catheter) that is associated with a lower risk of urinary tract infections. This recommendation can be tailored based upon the patient P's past and current medical state and the procedure being completed.

Further, the recommendation module 230 can provide recommendations for enhanced processes associated with the patient P based upon the risk assessment. For instance, if the patient P is at an elevated risk based upon the risk score, the recommendation module 230 can provide enhanced guidance for such processes, like cleaning and/or other perineal care (“peri care”). In such an example, the recommendation from the recommendation module 230 may include more frequent perineal cleanings or early removal of the catheter. Such recommendations can be tailored based upon the information for the patient P.

In another example, other processes can be recommended to further mitigate against the risk. For instance, with an elevated risk score and certain procedures, the recommendation module 230 can recommend ancillary processes, to mitigate the risk, such as the use of mitigation systems like the WatchCare™ Incontinence Management System from Hillrom. In another example, the PureWick™ urine collection system from Liberator Medical Supply, Inc. can be used to provide notifications related to proper placement, status, and removal of a catheter. Such systems can provide monitoring and notification when certain conditions are present.

Notifications can take various forms. For instance, the notification can simply be a warning like: “Warning; the patient is at an increased risk of urinary tract infections.” In another example, a recommendation can be provided: “It is recommended that an external catheter be used based upon an increased risk of urinary tract infections.” In yet another example, recommendations on enhanced procedures is provided: “It is recommended that the area surrounding the catheter be cleaned every 4 hours based upon an increased risk of urinary tract infections.”

Further, the notifications can be routed and graded according to severity to allow the caregiver to triage appropriately. For example, the notifications can be stratified from low, medium, and high to indicate the severity and response times necessary. Many other configurations are possible.

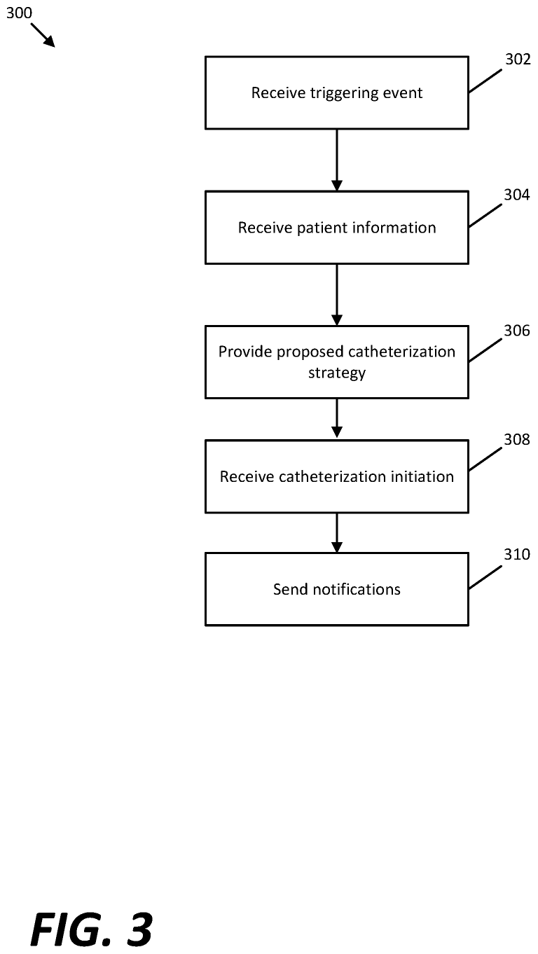

Referring now to FIG. 3, an example method 300 is shown. In this example, the server 150 is programmed to execute the method 300.

Initially, at operation 302, a triggering event is received for the catheterization of the patient. As described above, this triggering event can be automatically or manually generated.

Next, at operation 304, information about the patient is obtained. As described above, this information can be obtained from various sources, such as the EMR. At operation 306, a recommendation for catheterization is provided to the caregiver. For instance, as described above, the recommendation can simply be a notification of the heightened risk of a urinary tract infections and/or a recommendation of a different form of catheterization.

At operation 308, an indication of the start of the catheterization is provided. This can be an automated indication, such as by the controller 102. Alternatively, the caregiver can manually indicate the initiation of catheterization, such as by indicating the application of the catheter on the controller 102 or the mobile device 130.

Finally, at operation 310, notifications associated with the catheterization are sent. As noted, these notifications can be initiated by controller 102 and/or the server 150 in response to expiration of the countdown timer signifying the time for removal of the catheter.

Referring now to FIG. 4, an example user interface 400 is illustrated. In this example, the user interface 400 is generated on the mobile device 130 upon selection of the patient P. The user interface 400 displays information from the patient P's EMR. The network 20 can integrate the mobile device 130 with the EMR system 140 and controller 102 such that the user interface 400 displays data acquired from the controller 102 and the patient P's EMR.

The user interface 400 includes a bibliographic section 402 that identifies the patient P's name (e.g., “Hill, Larry”), medical record number (MRN) (e.g., “MRN: 176290”), date of birth (e.g., “DOB: Aug. 22, 1943”), age (e.g., “Age: 76”), sex (e.g., “Male”), identified risks (e.g., falls risk, pneumonia risk, injury risk, etc.), and primary diagnosis (e.g., “pneumonia”).

The user interface 400 includes a vital signs dashboard 404 that can display the patient P's recorded vital signs such as non-invasive blood pressure (NIBP), SpO₂, respiration rate, hear rate, temperature, and level of consciousness (LOC) (e.g., “lethargic”). Additionally, the vital signs dashboard 404 can display a modified early warning score (MEWS) assigned to the patient P that can include an arrow icon to indicate whether it is trending upwards or downwards, and a time stamp to indicate the last time it was updated.

The user interface 400 further includes a risk associated with catheterization section 406 that includes an example notification area 410. The risk associated with catheterization section 406 provides the caregiver with information associated with the catheterization of the patient P. For instance, the notification area 410 can provide a warning to the caregiver and/or a recommendation for a different catheterization process, as described above. Further, the notification area 410 of the risk associated with catheterization section 406 can allow the caregiver to interact with the server 150 to update the status of the patient P (e.g., indicate when the catheter is applied, or select an alternative catheterization process). Further, the notification area 410 can provide a notification for catheter removal. As described, such notifications can be snoozed or otherwise reassigned by the caregiver, as needed.

In some examples, the risk associated with catheterization section 406 is displayed automatically on the user interface 400 when the patient P is ordered to undergo a catheterization. When catheterization is not relevant to the patient P, the risk associated with catheterization section 406 can be hidden on the user interface 400.

The user interface 400 can further include a care communication section 408 that includes selectable icons that when selected display additional information related to the care of the patient P. For example, the selectable icons can include icons that when selected display the caregivers and care team members assigned to the patient P, the patient P's lab results, reminders on care protocols for the patient P, and alerts generated for the patient P.

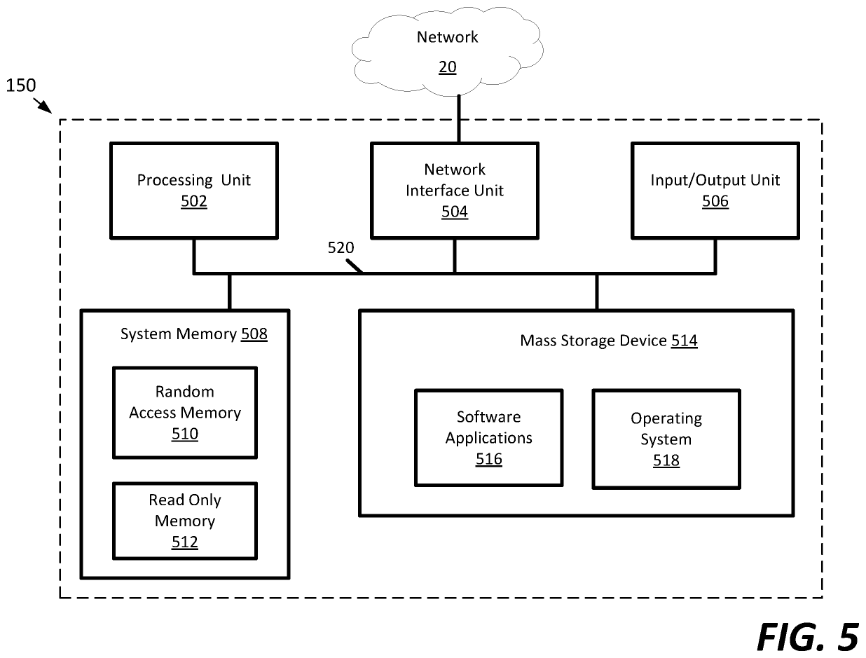

FIG. 5 schematically illustrates the server 150 used to implement aspects of the present disclosure. While the server 150 is shown, other computing devices that are part of the patient care system 10 can have similar components.

The server 150 has a processing unit 502, a system memory 508, and a system bus 520 coupling the system memory 508 to the processing unit 502. The processing unit 502 is an example of a processing device such as a central processing unit (CPU).

The system memory 508 is an example of a computer readable data storage device. The system memory 508 includes a random-access memory (“RAM”) 510 and a read-only memory (“ROM”) 512. Input/output logic containing the routines to transfer data between elements within the server 150, such as during startup, is stored in the ROM 512.

The server 150 can also include a mass storage device 514 that is able to store software instructions and data. The mass storage device 514 is connected to the processing unit 502 through a mass storage controller (not shown) connected to the system bus 520. The mass storage device 514 and its associated computer-readable data storage medium provide non-volatile, non-transitory storage for the server 150.

Although the description of computer-readable data storage media contained herein refers to a mass storage device, it should be appreciated by those skilled in the art that computer-readable data storage media can be any available non-transitory, physical device or article of manufacture from which the device can read data and/or instructions. The mass storage device 514 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The server 150 may operate in a networked environment using logical connections to remote network devices, including the mobile devices 130, EMR system 140, and the server 150, through the network 20. The server 150 connects to the network 20 through a network interface unit 504 connected to the system bus 520. The network interface unit 504 may also be utilized to connect to other types of networks and remote computing systems.

The server 150 can also include an input/output controller 506 for receiving and processing input from a number of input devices. Similarly, the input/output controller 506 may provide output to a number of output devices.

The mass storage device 514 and the RAM 510 can store software instructions and data. The software instructions can include an operating system 518 suitable for controlling the operation of the server 150. The mass storage device 514 and/or the RAM 510 also store software instructions 516, that when executed by the processing unit 502, cause the server 150 to provide the functionalities discussed in this document.

The various embodiments described above are provided by way of illustration only and should not be construed to be limiting in any way. Various modifications can be made to the embodiments described above without departing from the true spirit and scope of the disclosure.

What is claimed is:

1. A system for assessing risk associated with catheterization, the system comprising:
a processor; and
memory encoding instructions which, when executed by the processor, cause the processor to:
receive information relating to a current state of a patient;
perform an assessment of risk associated with the catheterization of the patient; and
provide a recommendation for the catheterization based upon the assessment of risk.

2. The system of claim 1, wherein the information includes demographic information about the patient.

3. The system of claim 1, wherein the information includes one or more of demographic information about the patient, a current medical state of the patient, and a type of procedure to be performed on the patient.

4. The system of claim 3, wherein the current medical state of the patient includes one or more of:
vital signs data from a vital signs monitor associated with the patient; and
incontinence information from a incontinence pad associated with the patient.

5. The system of claim 1, comprising further instructions which, when executed by the processor, cause the processor to calculate a risk score based upon the information about the patient.

6. The system of claim 5, wherein the risk score combines risks associated with two or more of the information about the patient.

7. The system of claim 5, comprising further instructions which, when executed by the processor, cause the processor to use the risk score to provide the recommendation for the catheterization.

8. The system of claim 1, wherein the recommendation for the catheterization includes a notification about risks associated with the catheterization.

9. The system of claim 1, wherein the recommendation for the catheterization includes an alternative catheterization option based upon the assessment of risk, wherein the alternative catheterization option includes an external catheterization.

10. The system of claim 1, comprising further instructions which, when executed by the processor, cause the processor to provide a notification for removal of a catheter associated with the catheterization upon expiration of the catheterization.

11. A method for assessing risk associated with catheterization, the method comprising:
receiving information relating to a current state of a patient;
performing an assessment of risk associated with the catheterization of the patient; and
providing a recommendation for the catheterization based upon the assessment of risk.

12. The method of claim 11, wherein the information includes demographic information about the patient.

13. The method of claim 11, wherein the information includes one or more of: dwell time; demographic information about the patient; a current medical state of the patient; and a type of procedure to be performed on the patient.

14. The method of claim 11, further comprising calculating a risk score based upon the information about the patient.

15. The method of claim 14, wherein the risk score combines risks associated with two or more of the information about the patient.

16. The method of claim 14, further comprising using the risk score to provide the recommendation for the catheterization.

17. The method of claim 11, wherein the recommendation for the catheterization includes a notification about risks associated with the catheterization.

18. The method of claim 11, wherein the recommendation for the catheterization includes an alternative catheterization option based upon the assessment of risk, wherein the alternative catheterization option includes an external catheterization.

19. The method of claim 18, wherein the recommendation includes perineal cleaning.

20. The method of claim 11, further comprising providing a notification for removal of a catheter associated with the catheterization upon expiration of the catheterization.
